(19) United States
(12) Patent Application Publication
(10) Pub. No.: US 2015/0342485 A1
(43) Pub. Date: Dec. 3, 2015

(54) SYSTEM AND METHOD FOR VISUALLY DETERMINING A PHYSIOLOGICAL SIGNAL THRESHOLD

(71) Applicant: Dräger Medical GmbH, Lubeck (DE)

(72) Inventors: Brian R. HERTEL, Boxford, MA (US); Brian MacDONALD, Townsend, MA (US)

(21) Appl. No.: 14/723,794

(22) Filed: May 28, 2015

Related U.S. Application Data
(60) Provisional application No. 62/005,312, filed on May 30, 2014.

(51) Int. Cl. A61B 5/044; A61B 5/0472; A61B 5/00; A61B 5/04

(52) U.S. Cl. CPC A61B 5/044; A61B 5/04012; A61B 5/0472; A61B 5/7475

(57) ABSTRACT

An apparatus, system and method for visually determining a physiological signal threshold are capable of providing a graphical user interface (GUI) useful for determining an electrocardiogram threshold parameter. The apparatus includes a physiological signal monitor, a display and a processor. The processor coupled to the display and is configured to receive, from a patient, at least one physiological signal and generate a graphical user interface (GUI) in the display. The GUI includes a first display portion configured to display a subset of the at least one physiological signal and to display a threshold parameter of the at least one physiological signal. The threshold parameter is displayed in the GUI superimposed on the subset of the at least one physiological signal.

### CROSS REFERENCE TO RELATED APPLICATIONS

This application claims the benefit of priority of U.S. Provisional Patent Application 62/005,312 filed May 30, 2014, the entire contents of which are incorporated herein by reference.

### TECHNICAL FIELD

The subject matter described herein relates to physiological signal monitoring systems and methods with a display and a processor to display one or more physiological signals for a clinician to detect features of interest based on a physiological signal threshold, and more particularly to a physiological signal monitor system, physiological signal monitor and physiological signal monitoring method such as for an electrocardiogram (ECG) display and evaluation based on an ECG threshold set to detect features of interest in the ECG.

### BACKGROUND

Electrocardiograms (ECGs) are commonly used in a clinical setting to monitor heart functions. ECGs measure the electrical signals of the heart and provide information regarding the heart rate, rhythm, and strength of the heart muscles. In order to study heart problems such as coronary heart disease, heart attacks, arrhythmias, heart failure, cardiomyopathy, congenital heart defects, heart valve disease, pericarditis and the like, physicians set an ECG threshold to detect features of interest in the electrocardiogram.

In the current state of the art, electrocardiogram signals are displayed according to the scale or gain requested by a health care provider/medical professional/clinician (user). To choose an ECG threshold a health care provider typically enters a number. In the current state of the art, while the user may be able to view the electrocardiogram and enter a threshold, the user may not have a clear indication of where the entered ECG threshold falls relative to the electrocardiogram signal. Therefore, in the current state of the art, a user may, in fact, incorrectly set the ECG threshold.

An accurate ECG threshold provides improved heart rate detection and QRS complex characterization. For example, an accurately selected ECG threshold would reduce the occurrence of false positive asystole classifications. Additionally, a higher threshold may reduce the probability of double counting of QRS complexes. Similarly, a lower threshold may help detect small QRS complexes caused by cardiovascular disease or due to body type. Consequently, it is critical for a health care provider to select an accurate ECG threshold.

### SUMMARY OF THE INVENTION

It is desirable to have physiological signal monitoring systems, methods, and physiological signal monitors in which a physiological signal threshold may be set based on a visual display. It would also be advantageous if the visually displayed physiological signal threshold (i.e., threshold, marker, signal processing condition or factor) is related to a visual display of the one or more physiological signal of interest.

According to the present invention, a system, method, and physiological signal monitor are provided which allow a health care provider to set a physiological signal evaluation threshold (physiological signal threshold) based on a visual display. The physiological signal threshold is visually displayed in conjunction with a display of a physiological signal of interest, particularly with the visual displayed threshold superimposed on the physiological signal.

An exemplary embodiment of the present invention comprises an electrocardiogram (ECG) monitor comprising a display and a processor coupled to the display. The processor is configured to receive at least one patient physiological signal and generate a graphical user interface (GUI) in the display. A first display portion of the GUI displays a subset of the at least one physiological signal and a threshold parameter, such that the threshold parameter is superimposed upon the subset of the at least one physiological signal. The GUI, may also include a second display portion configured to display the at least one physiological signal received from the patient.

Another exemplary embodiment of the present invention comprises a method for receiving at least one physiological signal from a patient at a physiological signal monitor, generating a GUI, displaying a subset of the received at least one physiological signal in a first display portion of the GUI, and displaying a threshold parameter superimposed on the subset of the received at least one physiological signal. The GUI may also include a second display portion configured to display the at least one physiological signal received from the patient.

The method may include displaying, by way of an ECG monitor as the physiological signal monitor, the at least one physiological signal in a second display portion of the GUI. The at least one physiological signal and the subset of the at least one physiological signal may be displayed in real time. The at least one physiological signal may comprise at least one signal representative of the electrical activity of the heart. The superimposed threshold parameter may be represented by a straight line and the subset of the at least one physiological signal may be a waveform.

The method may further comprise receiving the signal parameter setting from a user, displaying, by way of the ECG monitor, a signal parameter setting and an ECG threshold setting in a third portion of the GUI and determining the subset of the at least one physiological signal based on the signal parameter setting. The signal parameter setting may comprise at least one of an ECG waveform setting and an ECG lead setting corresponding to signals from one or more ECG leads. The threshold parameter setting received may be based on at least one of an ECG threshold setting input by a user, an ECG threshold setting from a memory and an ECG threshold setting modified from a previous ECG threshold setting based on changes in the signal parameter setting. The GUI may be updated to reflect changes in the ECG threshold setting.

According to another aspect of the invention, a physiological signal monitor system is provided comprising electrodes for connection with a patient and a physiological signal monitor connected to the electrodes and receiving one or more electrical signals or voltage readings from, or derived from, the electrodes corresponding to electrical activity from the patient. The monitor comprises a display and a processor coupled to the display and configured to receive one or more electrical signals or voltage readings and generate a graphical user interface (GUI) in the display. The GUI comprises a combined physiological signal and threshold display portion displaying a physiological signal corresponding to all or a part of the electrical signals or voltage readings or derived from all or a part of the electrical signals or voltage readings and displaying a set threshold parameter of the physiological signal superimposed on the physiological signal. The GUI further comprises a setting selection display portion to set the threshold parameter of the physiological signal.

The processor is configured to receive at least one physiological signal from a patient and generate a graphical user interface (GUI) in the display. The display may include, for example, a liquid crystal display (LCD), computer-type display, a touch-screen display or other displays common in the field of computers. The touch screen display may employ the GUI to display data and receive user input. Other displays employee the GUI to display data and to coordinate input via an input device. The input device may for example be a computer mouse or keyboard controlling a cursor to activate softkeys/softbuttons (software displayed actuation portions—buttons) of the GUI. A first display portion of the GUI displays a subset (one or more signal selected from a set of possible signals) of the at least one physiological signal (the set of available signals) and a threshold parameter, such that the threshold parameter is superimposed upon the subset of the at least one physiological signal. The GUI, may also include a second display portion configured to display the at least one physiological signal received from the patient.

Also in accordance with the invention is a method for receiving at least one physiological signal from a patient at an ECG monitor, generating a GUI, displaying a subset of the received at least one physiological signal in a first display portion of the GUI, and displaying a threshold parameter superimposed on the subset of the received at least one physiological signal. The GUI, may also include a second display portion configured to display the at least one physiological signal received from the patient. Accordingly, a health care provider is able to select an ECG threshold parameter value based on the visual representation of the threshold parameter superimposed upon waveform data.

In accordance with another aspect of the invention, a medical monitor such as an ECG monitor is provided comprising a display and processor. The processor receives physiological signals representative of a physiological parameter of the patient from medical devices such as an ECG device, non-invasive blood pressure measurement device, electroencephalogram, electromyograph (EMG), pulse oximeters, and the like. The monitor may include processing features to form a medical device or medical system. The monitor may receive electrical signals or voltage readings directly or indirectly from one or more electrodes configured to record electrical activity from a patient. The electrical signals or voltage readings may correspond to the electrical activity of a patient's heart. The monitor may calculate the difference in the electrical activity recorded from attached electrodes to create an ECG signal. An ECG signal is a physiological signal and is typically graphically presented as waveforms. Clinical professionals study the waveforms or graphical representation of ECG signals to perform improved heart rate detection and QRS complex characterization.

In addition to receiving at least one patient physiological signal, the processor is configured to generate a GUI in the display. Signals received by the processor and displayed in the GUI may be processed according to signal processing techniques commonly known in the field. The GUI may, for example, contain a first display portion configured to display a subset of the at least one physiological signal. The subset of the at least one physiological signal may include, for example, only physiological signals that are represented as waveforms. The subset of the at least one physiological signal may also include, for example, only ECG signals. The subset of the at least one physiological signal may also include, for example, ECG signals from particular leads (electrode pairs). The subset may also be signals that have been processed in a particular way, to provide particular information. The subset of physiological signals displayed in the first display portion may, for example, be accompanied by a signal label and a scale label, indicating the type of signal displayed and the scale the signal is displayed at, respectively. The subset of the physiological signals displayed in the first display portion may be live (displayed based on a real time signal feed and/or processing).

The GUI generated by the processor may also include, for example, a second display portion configured to display the at least one physiological signal received from the patient. The second display portion may display all of the physiological signals the processor receives from the patient. Alternatively, in addition to displaying the subset of physiological signals displayed in the first display portion, the second display portion may be configured to display both continuous and discrete patient data. Continuous data, such as ECG waveforms may be displayed live. Discrete data, such as a non-invasive blood pressure measurement may display the last recorded measurement alongside an indication of the timing of the last measurement. The signals representative of a physiological parameter of the patient may be represented in waveform, numerical values, diagrams, and the like. The signals displayed in the first display portion of the GUI may be live and less than two seconds old.

In addition to displaying a subset of the at least one physiological signal received from a patient the first display portion of the GUI is also capable of displaying a threshold parameter such that the threshold parameter is superimposed upon the displayed subset of the at least one physiological signal. The threshold parameter, may correspond, for example to an exact voltage level. The threshold parameter may be groups of voltage levels or more than one threshold parameter may be set for multiple analytical tasks or functions or for processing that considers several aspects of the one or more physiological signals received. The threshold parameter may be may be a function, a signal envelope or a threshold with values that vary over a signal course, e.g., the threshold value may be a function of time or even a function of some measured parameter.

The subset of the at least one physiological signal displayed in the first display portion is chosen according to a signal parameter setting. The signal parameter setting may include an ECG waveform setting and an ECG lead setting. The ECG lead setting indicates the leads to be displayed and the ECG waveform setting indicates the number of waveforms or physiological signals to be displayed in the first display portion of the GUI. The options available for the signal parameter setting may vary according to the signals being received by the processor from the attached medical devices. For example, if the electrocardiogram is using only 3 leads, the user is allowed to choose from the three leads. The signal parameter setting may be input by pressing buttons on a touchscreen device. Alternatively, the signal parameter setting may be input by a keyboard, mouse, or other device. The signal parameter setting may also be input by selecting options from a drop down menu, scroll bars, checkboxes, and the like. The signal parameter setting may be input by a health care provider, technician, or patient (user). The chosen signal parameters may be indicated by way of a label or a textbox displayed on the GUI.

The first display portion displays the subset of the at least one ECG signal according to the signal parameter setting. The waveforms chosen by the settings are used to create the subset of signals to be displayed. The waveforms may be grouped together to be displayed in a section of the first display portion. The displayed waveforms are of the same form as those present in the second display portion. The displayed waveforms in the second display portion of the GUI are also accompanied by a signal label and a scale label. Similar to the waveforms displayed in the first display portion of the GUI, the waveforms in the second display portion of the GUI may also be displayed live (i.e., in real time).

The GUI may also comprise an area for a user to input a threshold setting. The user may choose the threshold setting by pressing a button corresponding to preconfigured threshold values. For example, for an ECG threshold, there may be two buttons labeled ‘High’ and ‘Normal’ which are preset to correspond to 0.4 mV and 0.17 mV, respectively. The ‘High’ and ‘Normal’ values may also be preset to correspond to higher or lower values. Alternatively, the ECG threshold setting may be input by entering a number into a textbox, scrolling through a range of values, depressing radio buttons and the like. The chosen ECG threshold value or threshold parameter is then converted to a graphical form such as a line, arrow, dotted line, curve, and the like. The graphical threshold parameter is then superimposed over the waveforms in the first display portion at the appropriate scale. For example, a graphical threshold parameter represented as a straight line may be horizontally oriented and superimposed upon a waveform such that both the waveform and the threshold may be viewed at the same time on the same figure. The graphical threshold parameter is updated automatically when the ECG threshold value is changed, thus allowing a health care provider to view the effect of changing the ECG threshold value visually.

The ECG threshold set by the user may be stored on a memory component. Additionally, the ECG waveform setting, and ECG lead setting may also be stored on a memory component. Alternatively, a stored ECG threshold setting, a stored ECG waveform setting, and a stored ECG lead setting may be pre-loaded into the GUI. The memory component may be coupled to the processor and/or the attached medical device. The selected ECG threshold may be transmitted to other cardiac monitoring devices, and/or other algorithms for use in cardiac feature detection and/or the patient's medical files. The GUI may also include a separate display portion in which the ECG threshold setting, the ECG waveform setting and the ECG lead setting may be input. The ECG threshold setting, the ECG waveform setting and the ECG lead setting may be grouped together to form a control area which spans a third portion of the GUI. The control area may include a section for viewing and changing the ECG threshold setting and the signal parameter setting (including the ECG waveform setting and ECG lead setting). An area of the display portion dedicated to choosing the signal parameter setting may be located adjacent to the area of the display dedicated to choosing the threshold setting. Alternatively, the threshold setting and signal parameter setting may be located a distance apart from each other. In yet another alternative, the signal parameter setting and threshold setting may form one or more separate display portions of the GUI.

The subset of the signals displayed in the first display portion may be resized according to the number of chosen waveforms and the size of the GUI. For example, the waveforms may be scaled to cover half of the display screen or half of the first display portion of the GUI. Any changes to the scale, sizing, or location of the first display portion of the GUI may result in automatic updates to one or more signal labels, and scale labels.

The first display portion of the GUI may be displayed upon selecting a specific tab from a menu. Alternatively, the first display portion of the GUI may be displayed on the monitor at all times.

As described above, the described GUI is implemented by the processor on a monitor using signals from an electrocardiogram device or other medical device or the monitor is configured to include the functionality of an ECG and/or EMG device and forms an electrocardiogram system in combination with electrodes. The monitor may be a stationary unit located at the bedside of a patient, while the processor and/or electrocardiogram device are mobile. Alternatively, the processor, monitor and electrocardiogram may all be mobile devices. The processor may form the electrocardiogram device (in combination with electrodes) and be integrated with the monitor or all or a part of the ECG processing function may be provided separate from the monitor.

In another alternative, the processor and electrocardiogram may be located with a patient while the monitor is located remotely. The flow of data and information between the processor, monitor, and electrocardiogram may be conducted through wired or wireless connections. The disclosed GUI may also be incorporated into an online viewing application used by physicians remotely.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which embodiments of the invention are illustrated.

### BRIEF DESCRIPTION OF THE DRAWINGS

In the drawings:

FIG. 1 is a screen shot view illustrating a touch screen display having a graphical user interface display features of an ECG monitor;

FIG. 2A illustrates the touch screen display of the ECG monitor showing a first threshold setting displayed superimposed on an ECG signal;

FIG. 2B illustrates the touch screen display of the ECG monitor showing a second threshold setting displayed superimposed on an ECG signal;

FIG. 3 is a schematic view showing a physiological signal monitor system;

FIG. 4 is a flow diagram showing aspects of a method; and

FIG. 5 is a flow diagram showing aspects of another method.

### DETAILED DESCRIPTION

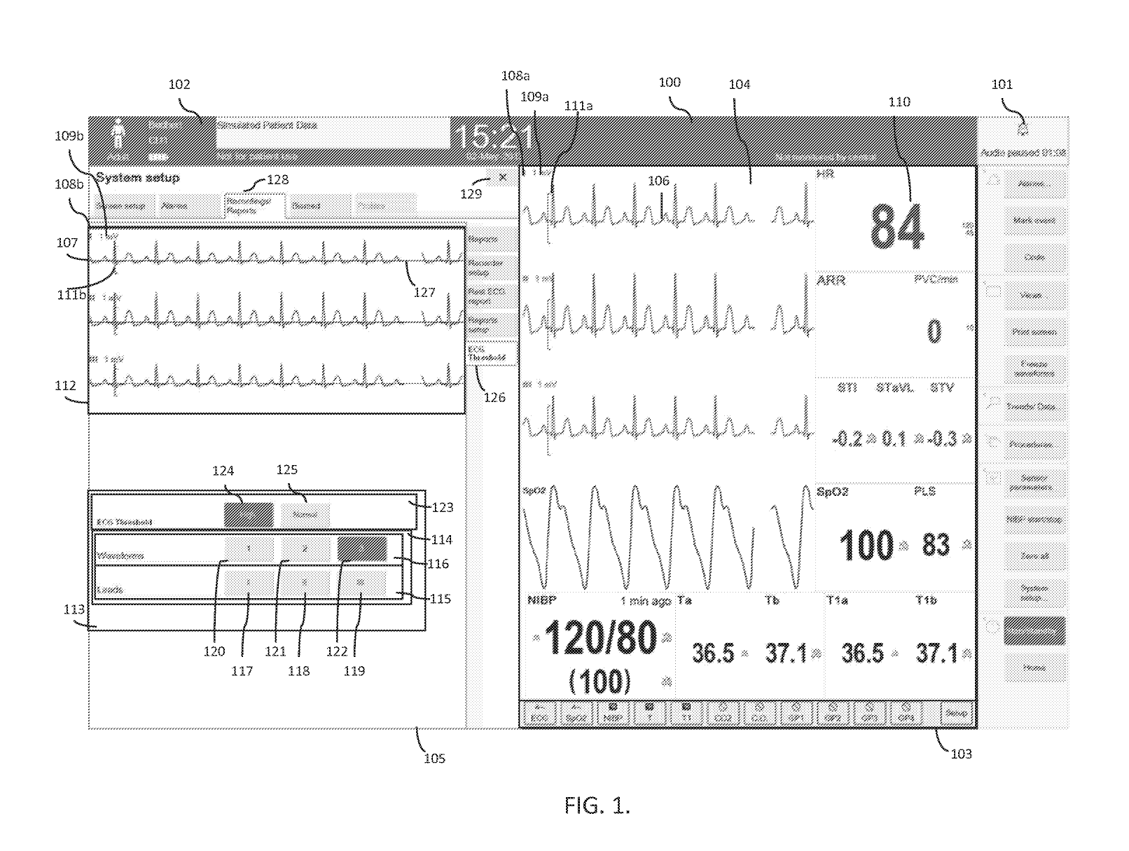

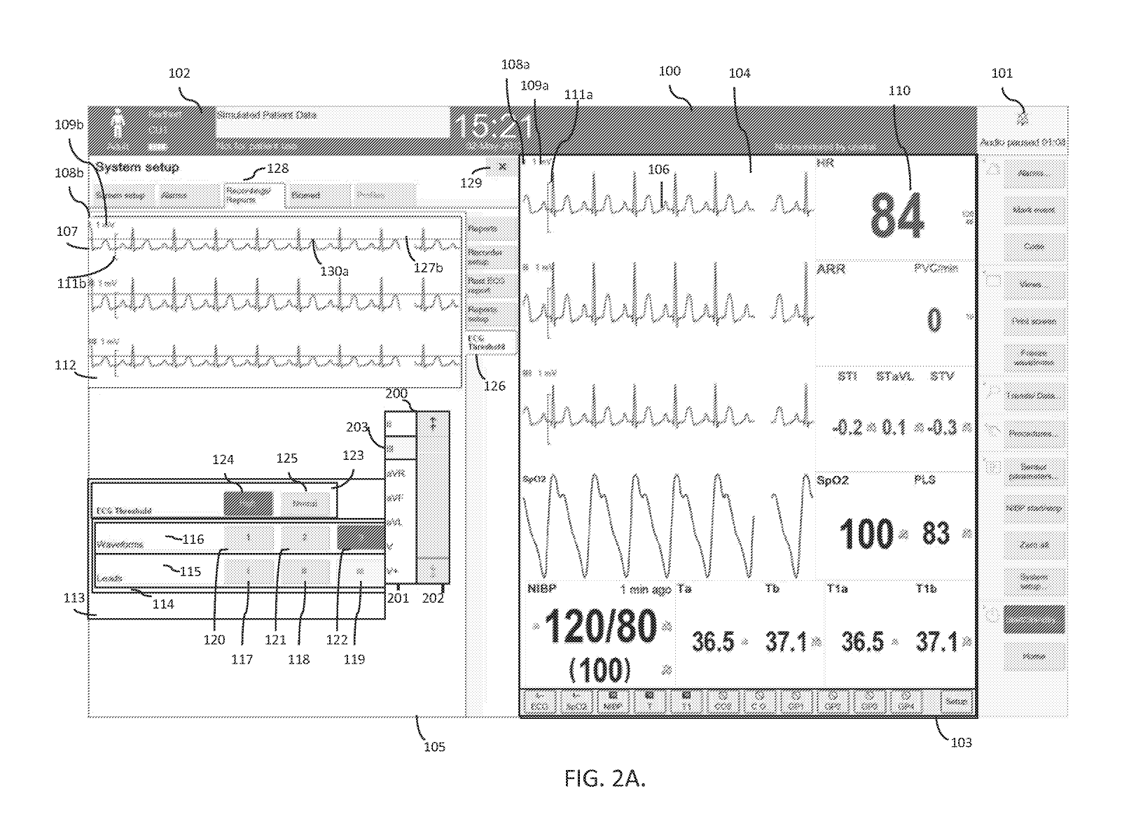

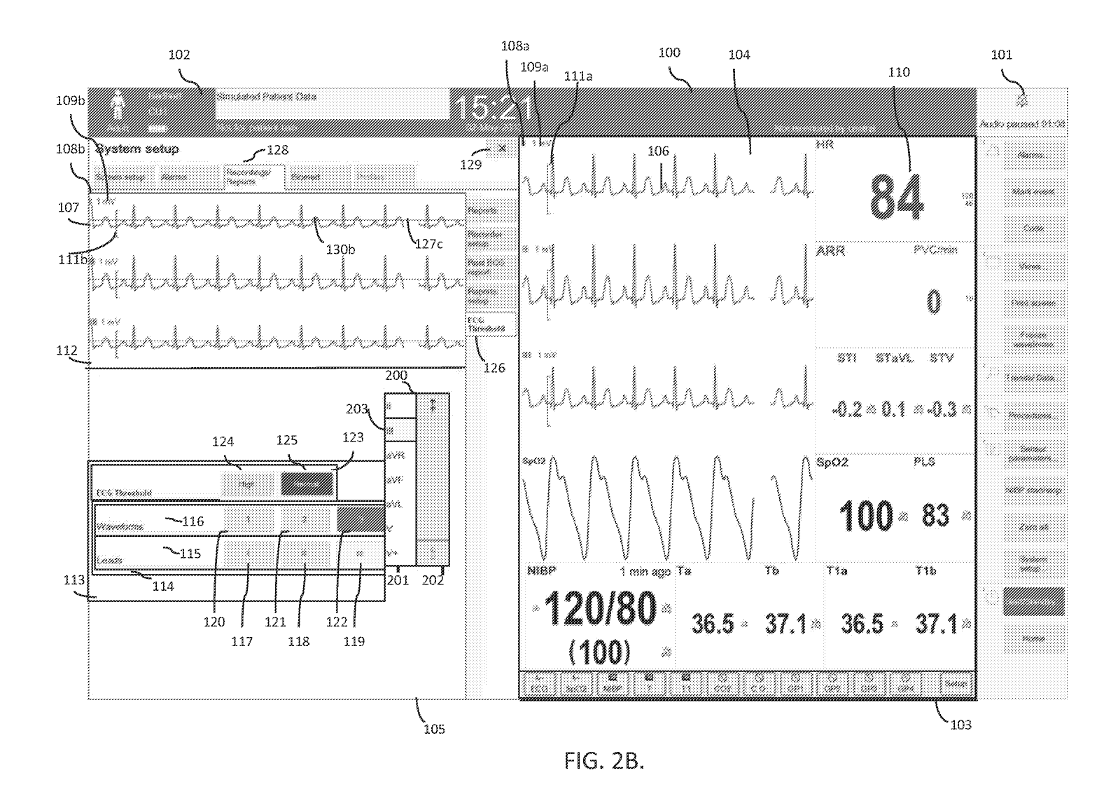

Referring to the drawings, FIG. 1 shows a GUI generally designated 100 including a first display portion 105 (a combined physiological signal and threshold display portion), in which a ECG threshold parameter of the physiological signal 127 is displayed superimposed over a patient physiological signal (waveform) 107. The GUI 100 includes a second display portion 104 with displayed waveforms 106, and a third display portion (setting selection display portion) or control area 113 for a selection of settings for the monitor 12. FIG. 2A shows the GUI 100 with a medical professional/clinician (user) selected ‘high’ value for an ECG threshold parameter 127 b and FIG. 2B shows the GUI 100 with a user selected ‘normal’ value for an ECG threshold parameter 127c.

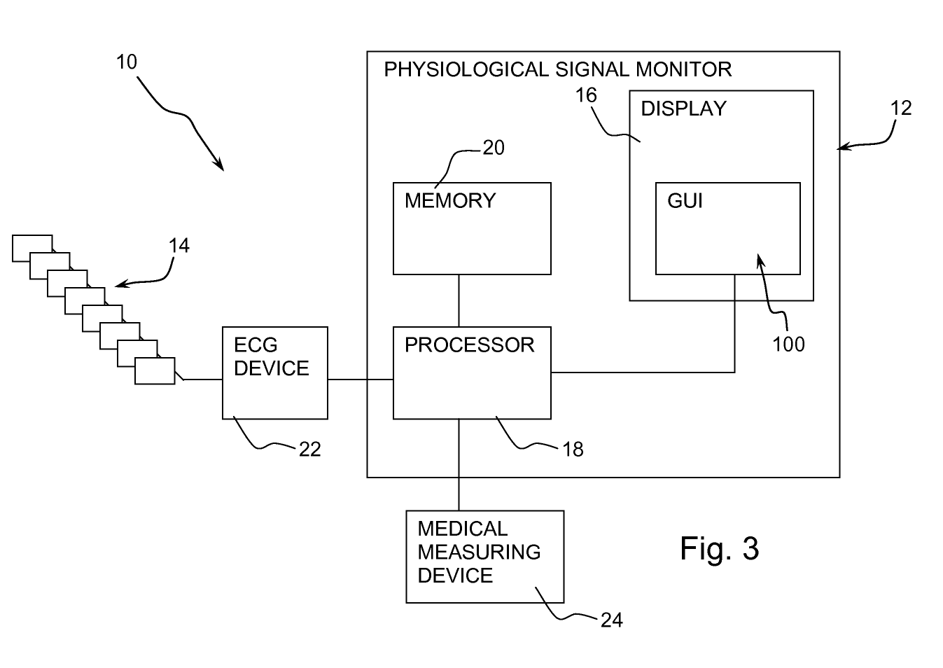

FIG. 3 shows a medical monitor system generally designated 10. The system 10 includes a physiological signal monitor in the form of an ECG monitor 12. The monitor 12 is connected to an electrode arrangement 14 via an ECG device 22. The ECG monitor 12 may also be provided as an ECG device (with all of the functionality of an ECG device including providing interpretive ECG machine functions and non-interpretive ECG machine functions). The monitor 12 may also be connected to one or more other medical measuring devices 24.

The monitor 12 includes a display 16 with the GUI 100. The electrodes 14 of the system 10 are two or more electrodes that sense electrical activity from a patient and provide signals to the ECG device and/or to the monitor 12. The monitor 12 includes the display 16 and a processor 18, coupled to the display and configured to receive one or more signals related to sensed electrical activity of the patient and for generating the GUI 100, including generating a display of one or more physiological signals at the GUI 100 of the display 16. The monitor 12 may also include a memory 20.

The number of electrodes is not essential. A particular pair of the electrodes 14 has a signal output which may differ from the output produced by other pairs, wherein the pairs (and/or signals from the pairs) are also referred to as ECG leads or signal leads. With more than one pair of electrodes (with plural leads), a set of physiological signals is provided by the system 10 for display by the display 16. A selected subset of the physiological signals may be displayed at the GUI 100, such as selected waveforms provided by or derived from the various leads.

The processor 18, or an external processor, may be configured with hardware or firmware or may be configured to cooperate with software to detect features of interest in one or more of the set of physiological signals based on the selected parameter threshold. The user uses the settings selection display portion or control area 113 to input a threshold parameter 127 of the physiological signal 107. The user is provided with immediate feedback as to whether the threshold 127 is appropriate to detect features of interest or to provide the necessary analysis. The feedback is provided based on the selected threshold parameter 127 being displayed in the GUI 100 superimposed on the physiological signal (waveform) 107. The user can see if the setting is appropriate for the processor and/or software to detect features of interest in the waveform by visually comparing the selected threshold parameter 127 with the physiological signal 107. If only what appear to be the peaks of QRS complexes, and not other ECG features such as T waves (see further description below), rise above (exceed) the selected threshold parameter 127, then the threshold parameter 127 is appropriately selected. For example, where the waveform 107 is an ECG signal from one or more of the leads of the electrodes 14, the user can determine if the threshold parameter 127 is set appropriately to correctly detect only peaks of QRS complexes. Based on this immediate feedback, via the graphical user interface 100, the user can make an appropriate adjustment to the setting selection display portion 113 to change or adjust the threshold parameter 127 of the physiological signal 107.

In some embodiments, the processor 18 (or an external processor) can be used to detect QRS complexes in the ECG signal that exceed the threshold parameter 127 during a pre-determined time interval. In such embodiments, the processor 18 may perform analysis on the detected QRS complexes for heart rate detection and QRS complex characterization (e.g., normal, premature ventricular contraction (PVC), premature atrial contractions (PAC), atrial fibrillation, atrial flutter, paroxysmal supraventricular tachycardia (PSVT), accessory pathway tachycardias, AV nodal reentrant tachycardia, ventricular tachycardia (V-tach), ventricular fibrillation, long QT syndrome, bradyarrhythmias, sinus node dysfunction, and heart block).

The second display portion 104 of the GUI 100 is displayed on the right side of the display 16 of the monitor 12. In this section a plurality of signals indicating the physiological state of the patient are shown. This may be the main display portion with continuous physiological signals displayed as a waveform 106 and discrete physiological signals as a numerical value 110. The first display portion 105 of the GUI 100 is shown on the left side of the monitor. The waveform 107, corresponding to one of the waveforms 106 displayed in the second display portion 104, is displayed on the first display portion 105. As the corresponding waveforms are identically displayed the corresponding signal labels 108a and 108b are also identical. Scale labels are comprised of text (e.g., 1 mV) 109a and 109b and a vertical bracket 111a and 111b. In this embodiment each of the scale labels 109a, 111a and 109b, 111b correspond to the same waveform such that they may be identical. If they are not identical, they are adjusted according to the size of the waveform displayed.

The subset of at least one signal representative of a physiological parameter of a patient, displayed in the first display portion 105 of the GUI 100, may be grouped together in a waveform viewing area 112. The subset may be e.g., the ECG waves from selected leads (electrode pairs)—in which the set is all of the waves from all of the available leads. The settings pertaining to the waveforms 107 displayed and threshold value 127 set (and superimposed on the respective waveforms 107 displayed) may also be grouped together, for example in the control area 113. The control area 113 may form the third portion of the GUI. The third portion of the GUI may be located adjacent to or within the first display portion 105. The control area 113 further comprises a signal parameter setting area 114 and an ECG threshold setting area 123. The signal parameter setting area 114 may comprise a lead setting area 115 and a waveform setting area 116. In this example, the lead setting area further comprises buttons (softkeys) which control the leads to be displayed in the waveform viewing area 112. The contents of the buttons 117-119 are adjusted according to the lead sets used by the electrocardiogram system 10. For example, only those lead sets which are currently transmitting information to the processor will be selectable by the user. Similarly, in this example, the waveform setting area also comprises buttons 120-122. These waveform setting area buttons 120-122 allow the user to select how many ECG waveforms will be displayed in the waveform viewing area 112 of the first display portion 105 of the GUI 100. The number of waveforms to be displayed may be represented as button labels. A selection of the number of waveforms may be indicated by shading 122. The ECG threshold setting area 123 is also included in the control area 113. As shown, the ECG threshold setting area 123 contains buttons 124 and 125 which are preconfigured to correspond to a preset threshold value. In this example, when the ‘High’ button is chosen a threshold line 127 of 0.4 mV is superimposed upon the waveforms 107 (or on selected waveforms) in the first display portion 105 of the GUI 100. In this example, when the ‘Normal’ button is chosen a threshold line of 0.17 mV is superimposed upon the waveforms 107 (or on selected waveforms) in the first display portion 105 of the GUI 100. The waveforms 107 displayed in the waveform viewing area 112 are automatically updated along with the signal label 108b, scale label 109b, and threshold line 127 according to the choices selected by the user in the control area 113. The first display portion 105 of the GUI 100 is displayed upon selecting an ECG threshold tab 126, which is available upon selecting a Recordings/Report tab 128. Additionally, the first portion 105 of the GUI 100 can be removed from display by selecting an exit feature 129. This allows the second portion 104 to occupy most of the GUI 100 of the display 16.

FIG. 2A shows the same GUI as in FIG. 1. However, the user has selected different settings. For example, in FIG. 2A the user has selected a ‘High’ value for the ECG threshold, accordingly the ECG threshold line 127b is placed higher in the waveform viewing area 112. Also shown in FIG. 2A, is a drop down menu 200 for selecting the ECG lead setting. The user uses the drop down menu 200, in which the user may use scroll feature 202, to view lead options 201. In this example, the user has the option of selecting from a set of signal leads typically known in the field. Alternatively, the user may have the option to select an ECG lead (signal lead) setting in a different menu-style such as a rotary wheel, checkboxes, textboxes, and the like. The selected lead 203 may be highlighted within the drop down menu 200. The selected lead 203 may provide a label for the corresponding lead configuration button 119. Additionally, the selected lead 203 may provide a signal label for the waveform viewing area 112.

FIG. 2B shows the same GUI 100 as in FIG. 1 and FIG. 2A. However, the user has selected different settings. For example, in FIG. 2B the user has selected a ‘Normal’ value for the ECG threshold, accordingly the ECG threshold line 127c is placed lower in the waveform view in area 112.

The importance of selecting the correct threshold parameter is evident from a comparison of FIGS. 2A and 2B. In contrast to FIG. 2A, FIG. 2B shows the ECG T-waves 130b rising above the ‘Normal’ threshold 127c, while in FIG. 2A the ECG T-waves 130a are below the ‘High’ threshold 127b. If the user uses the ‘Normal’ threshold, as shown in FIG. 2B, errors in QRS detection may occur, because the T-waves may be incorrectly classified as QRS complexes. Accordingly, the user is able to select the appropriate ECG threshold by viewing the threshold parameter superimposed upon the waveforms.

As shown in FIGS. 1, 2A and 2B the GUI 100 may include additional features such as menu buttons 101, a header 102, device indicators 103 and the like. Menu buttons may include buttons for an alarm, marking event, code, views, print screen, freeze waveforms, trends/data, procedures, sensor parameters, NIBP start/stop, zero all, system setup, start/standby, home, and the like. The buttons may serve to start/stop medical devices connected to the monitor, adjust how items are displayed in the GUI, initiate the creation of additional portions of the GUI and the like. For example, choosing an ECG threshold button from a menu may result in displaying the first display portion. A header section may include patient information such as age, weight, gender, name, primary physician, risk factors and the like. The header may also include, for example, an indication of the monitoring unit to which a portable monitor is transmitting, a battery status, the time and date, and a sound/alarm indicator. A device indicator included in the GUI may indicate the devices from which the processor is capable of receiving physiological signals from.

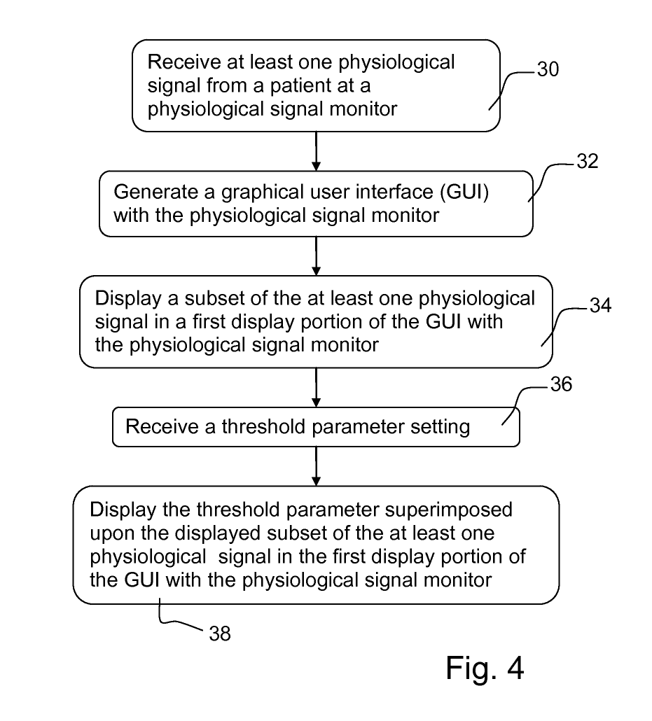

FIG. 4 shows method steps of a physiological signal monitor method. As shown at step 30, the method includes receiving at least one physiological signal from a patient at a physiological signal monitor 12. As discussed above, the monitor 12 may be an ECG monitor that includes or is connected to an ECG device 22. The ECG device 22 provides the at least one physiological signal to the monitor 12, based on signals from surface electrodes 14. The graphical user interface (GUI) 100 is generated with the physiological signal monitor 12 at step 32. The GUI 100 displays a subset of the at least one physiological signal in a first display portion 105 at step 34. A threshold parameter setting is then received at step 36. This may be an input of a threshold parameter using the control area 113 of the GUI 100. At step 38 the threshold parameter is displayed superimposed upon the displayed subset of the at least one physiological signal in the first display portion 105 of the GUI 100 with the physiological signal monitor 12.

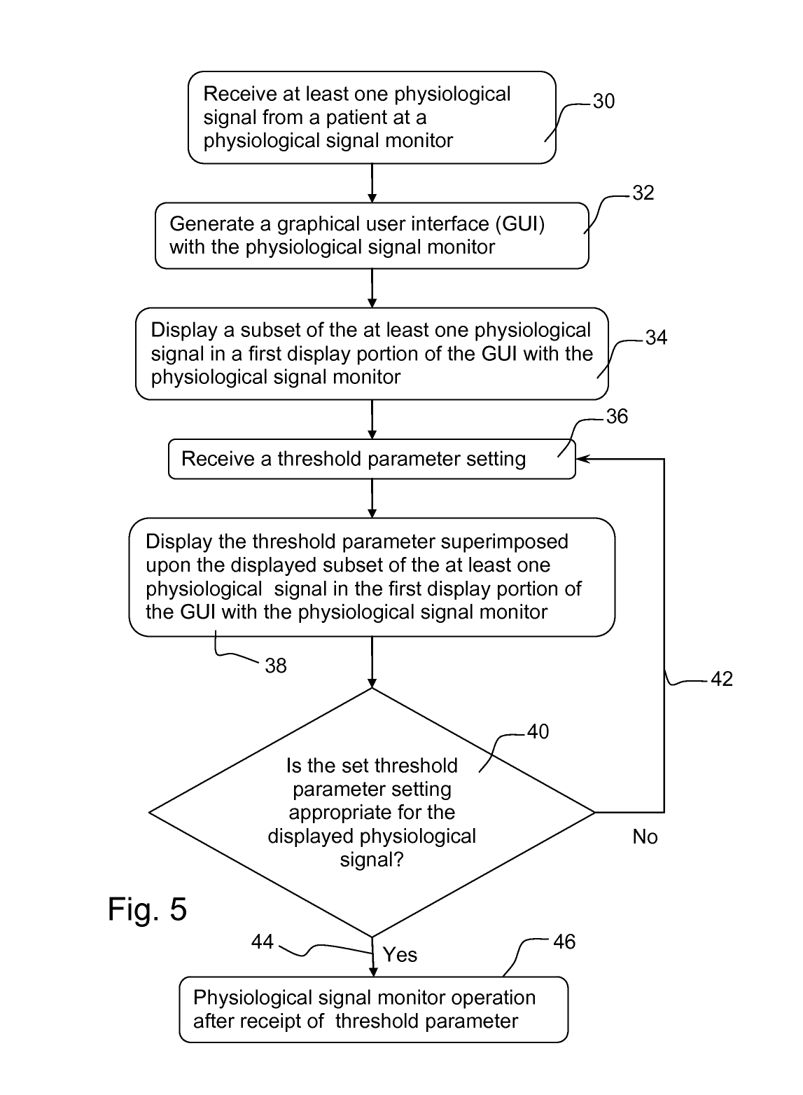

FIG. 5 shows method steps of another physiological signal monitor method. The method includes the step 30 of receiving at least one physiological signal from a patient at a physiological signal monitor 12. As discussed above, the monitor 12 may be an ECG monitor that includes or is connected to the ECG device 22. The graphical user interface (GUI) 100 is generated with the physiological signal monitor 12 at step 32. The GUI 100 displays a subset of the at least one physiological signal in a first display portion 105 at step 34. A threshold parameter setting is then received at step 36. This may be an input of a threshold parameter using the control area 113 of the GUI 100. At step 38 the threshold parameter is displayed superimposed upon the displayed subset of the at least one physiological signal in the first display portion 105 of the GUI 100 with the physiological signal monitor 12. The method of FIG. 5 may continue at step 40 with it being considered, based on the display of the threshold parameter superimposed upon the physiological signal, if the set threshold parameter is appropriate for the displayed physiological signal. If threshold parameter setting is appropriate, the method continues with the yes branch 44. The physiological signal monitor operates, after the threshold parameter is received, as shown at step 46. If it is determined that the threshold parameter setting is not appropriate, the method continues with the no branch 42. The method again proceeds with step 36, with a different threshold parameter being set, and with step 38. The newly set threshold parameter is displayed superimposed upon the displayed subset of the at least one physiological signal in the first display portion 105 of the GUI 100.

Although embodiments of the present invention have been described above for use in setting an ECG threshold using a visual display, those of ordinary skill in the art will realize that the embodiments of the present invention may be used for other types of operations and procedures such as spike detections in electroencephalograms and other related clinical techniques which would benefit from displaying a threshold or similar marker superimposed over a clinical signal.

While specific embodiments of the invention have been shown and described in detail to illustrate the application of the principles of the invention, it will be understood that the invention may be embodied otherwise without departing from such principles.

What is claimed is:

1. A physiological signal monitor comprising:
a display; and
a processor coupled to the display and configured to receive at least one patient physiological signal and generate a graphical user interface (GUI) in the display comprising:
a first display portion configured to display a subset of the at least one physiological signal and to display a threshold parameter of the at least one physiological signal, wherein the threshold parameter is displayed in the GUI superimposed on the subset of the at least one physiological signal.

2. A physiological signal monitor according to claim 1, wherein the GUI in the display further comprises a second display portion configured to display the at least one physiological signal received from the patient.

3. A physiological signal monitor according to claim 1, wherein the at least one physiological signal and the subset of the at least one physiological signal are displayed in real time.

4. A physiological signal monitor according to claim 1, wherein the at least one physiological signal comprises at least one signal representative of the electrical activity of the heart.

5. A physiological signal monitor according to claim 4, wherein at least one signal representative of the electrical activity of the heart is comprised of an ECG signal and the threshold parameter is established with an ECG threshold setting interface.

6. A physiological signal monitor according to claim 1, wherein the GUI in the display further comprises a third display portion configured to display a signal parameter setting interface and a threshold setting interface.

7. A physiological signal monitor according to claim 6, wherein the subset of the at least one physiological signal is selected with a GUI softkey or a GUI displayed input for the signal parameter setting interface.

8. A physiological signal monitor according to claim 7, wherein the signal parameter setting interface further comprises a waveform setting interface.

9. A physiological signal monitor according to claim 7, wherein the signal parameter setting interface further comprises a lead setting interface to select signals from one or more signal leads.

10. A physiological signal monitor according to claim 1, further comprising: a memory coupled to the processor.

11. A physiological signal monitor according to claim 10, wherein the memory stores one or more settings related to the GUI.

12. A physiological signal monitor according to claim 1, wherein the display is a touchscreen display capable of receiving user input.

13. A physiological signal monitor according to claim 1, wherein the superimposed threshold parameter is represented by a straight line and the subset of the at least one physiological signal is a waveform.

14. A physiological signal monitor system comprising:
electrodes for connection with a patient; and
a physiological signal monitor connected to the electrodes and receiving one or more electrical signals or voltage readings from, or derived from, the electrodes and corresponding to electrical activity from the patient, the monitor comprising:
a display; and
a processor coupled to the display and configured to receive one or more electrical signals or voltage readings and generate a graphical user interface (GUI) in the display comprising:
a combined physiological signal and threshold display portion displaying a physiological signal corresponding to all or a part of the electrical signals or voltage readings or derived from all or a part of the electrical signals or voltage readings and displaying a threshold parameter of the physiological signal superimposed on the physiological signal; and
a setting selection display portion to set the threshold parameter of the physiological signal.

15. A medical monitor system according to claim 15, wherein:
the physiological signal is an ECG signal; and
the processor detects QRS complexes in the ECG signal that exceed the threshold parameter during a pre-determined time interval.

16. A medical monitor system according to claim 16, wherein:
the processor performs analysis on the detected QRS complexes for at least one of heart rate detection and QRS complex characterization.

17. A method comprising:
receiving, at a physiological signal monitor, at least one physiological signal from a patient;
generating, by way of said physiological signal monitor, a graphical user interface (GUI);
displaying, by way of said physiological signal monitor, a subset of the at least one physiological signal in a first display portion of the GUI;
receiving a threshold parameter setting;
displaying, by way of said physiological signal monitor, the threshold parameter in the first display portion of the GUI, wherein the threshold parameter is superimposed upon the displayed subset of the at least one physiological signal.

18. The method of claim 17, wherein the at least one physiological signal and the subset of the at least one physiological signal are received and displayed in real time.

19. The method of claim 17, wherein the at least one physiological signal comprises at least one signal representative of the electrical activity of the heart comprised of an ECG signal and the threshold parameter is established with an ECG threshold setting interface.

20. The method of claim 17, wherein the superimposed threshold parameter is represented by a straight line and the subset of the at least one physiological signal is a waveform.
